(19) United States
(12) Patent Application Publication
(10) Pub. No.: US 2002/0172960 A1
(43) Pub. Date: Nov. 21, 2002

(54) DNA microarrays of networked oligonucleotides

(76) Inventors: Yijia Bao, Painted Post, NY (US); Ann M. Ferrie, Painted Post, NY (US); Fang Lai, Painted Post, NY (US); Thomas L. Volkert, Cambridge, MA (US); Xinying Xie, Painted Post, NY (US); Qin Zong, Germantown, MD (US)

Correspondence Address: CORNING INCORPORATED, SP-TI-3-1, CORNING, NY, 14831

(21) Appl. No.: 10/028,512

(22) Filed: Dec. 20, 2001

Related U.S. Application Data
(63) Continuation-in-part of application No. 09/859,752, filed on May 17, 2001.

(51) Int. Cl. C12Q 1/68; C12M 1/34

(52) U.S. Cl. 435/6; 435/287.2

(57) ABSTRACT

A method for producing a DNA microarray for biological assays comprising a plurality of biosites located on a solid substrate. Each biosite having at least one set of networked oligonucleotides of medium to long lengths attached to the substrate by predominantly non-covalent means. The oligonucleotide strands are chemically presynthesized, exhibit no predetermined spatial orientation on the substrate surface, and have three-dimensional freedom of movement when in a hybridization solution. The oligonucleotides are chemically unmodified having a length of about 30 bases or longer. Preferably, the oligonucleotides are from about 45 bases to 85 bases, but some embodiments may include very long oligonucleotides of over 100 bases.

### RELATED APPLICATIONS

This Application is a Continuation-in-Part of U.S. patent application Ser. No. 09/859,752, filed on May 17, 2001, in the names of Vickesh E. Myer, David G. Statham, Thomas L. Volkert, and Christopher J. Wilson, the entire content of which is incorporated herein by reference.

### FIELD OF THE INVENTION

The invention relates to the field of molecular biology, and more particularly to the field of assays involving nucleic acid hybridization and the use of so-called “DNA-chip” or microarray technology. The invention provides a method for making and using array-based nucleic acid hybridization substrates.

### BACKGROUND

DNA-microarray technology has broad applications in genomic and pharmaceutical research, as well as medical diagnostics, which include gene discovery, gene expression monitoring, polymorphism screening, drug discovery, and clinical trial monitoring. DNA-microarrays provide a means to quantify tens of thousands of discrete genetic sequences in a single assay. Based on the use of such arrays of nucleic acid probes on solid surfaces, large-scale analysis of gene expression and mutations are becoming a reality. A key to these advances has been the development of immobilization chemistry for the spatially resolved attachment of DNA probes to a solid support surface.

Recent research in the microarray field has concentrated on the development of covalent coupling of relatively short oligonucleotides probes to planar surfaces. Such covalent coupling requires activation of the underlying planar surface with cross-linking reagents and/or modifications of the DNA molecule with a reactive group. At present, alternatives using non-covalent approaches have not been particularly successful. Some have suggested that DNA probe molecules attached to the surface of a substrate by multiple constraining contacts would serve as poor hydridization probes, because of the loss of configurational freedom and the attendant loss of capacity to form bonds between strands of targets and probes. Moreover, some have pointed out that DNA molecules attached non-covalently would be susceptible to removal from the substrate surface, if only a few contacts were made between the nucleic probe and surface. Therefore, the field of DNA-microarray using oligonucleotides has focused most of its attention on covalent attachment strategies.

A critical, but often time-consuming and costly step in the production of DNA microarrays is the up-front procurement and selection of high quality DNA content. Two major types of platforms for manufacture of high-density microarrays have been developed. The first platform involves relatively short (≦25 bases) oligonucleotides made by a photolithographic process similar to the manufacture of computer chips. This type of fabrication is often a multi-stepped process that involves in-situ synthesis of oligonucleotides on a solid substrate, such as a slide. The second platform uses robotic deposition or “spotting” of DNA molecules onto a specially coated substrate. Spotted arrays are commonly referred to as “cDNA microarrays,” although clones, PCR amplified products, or oligonucleotides (pre-synthesized) can all be spotted onto a specially coated substrate. To provide the linkage and spacer elements for attaching an oligonucleotide to a slide surface, a pre-synthesized oligonucleotide is usually chemically modified by the addition of a functional group to either its 5′ or 3′ end.

Each of these two approaches for microarray designs possesses a number of virtues. For example, on one hand, arrays that use PCR-product sequences tend typically to exhibit a better signal than devices with printed oligonucleotides. Oligonucleotides arrays, on the other hand, offer greater specificity than cDNAs, including PCR products, having the capability to distinguish single nucleotide polymorphisms and discern splice variants. In other words, oligonucleotide arrays are independent of cDNA templates, and their relatively short length avoids repetitive homologues of PCR amplified nucleic acid sequences, which may lead to problems of cross hybridization and high background signal.

More importantly, however, are the shortcomings of each platform. Sequencing efforts have greatly enlarged the bank of useful information available for microarray products, yet, for these diverse products the potential has not been realized using PCR amplified DNA. PCR, while useful for producing sizeable quantities of genetic sequences, is nonetheless DNA template dependent, thus subject to limitations in the potential variations of genetic expression. PCR forces an array manufacturer to select DNA content based on publicly or privately available DNA in the form of cDNA clones, genomic DNA primers, or PCR amplifed templates. Moreover, PCR can become a bottleneck during product development or manufactured when template sources, such as EST clones, full-length cDNA, or other DNAs, becomes unavailable. For example, microarray product concepts such as toxicology arrays or bacterial genome arrays without template sources cannot be manufacture until original template sources are identified or made.

Although not hindered by the availability of templates like PCR products, relatively short oligonucleotides (≦25-mer) on arrays, in contrast, do not provide the requisite level of signal for improved imaging, nor do they bind well to sample target nucleic sequences during hybridization because of steric problems associated with their short lengths and the secondary structures of labeled targets. Also, even presynthesized oligonucleotides that are chemically modified have a drawback. These oliogonucleotides, as a consequence of being chemically modified, require special surfaces for attachment, and may lessen performance and specificity. The modified oligonucleotides tend to function less naturalistically than unmodified oligonucleotides when binding to target nucleic sequences. Moreover, in both systems, creation of a new array design is relatively inconvenient and/or expensive, requiring either a new set of masks for photolithography, or new samples to deposit for spotted cDNA arrays. Flexibility to create new arrays is becoming increasingly important as more genomes are sequenced and more applications for microarrays are described.

Hence, an invention which can combine the advantages of both PCR and oligonucleotide microarrays, and provide a viable, non-covalent adsorption to a planar substrate would likely receive a warm welcome from workers in the biological, medical, or pharmaceutical fields. The present invention meets such a need. The present invention provides a design for and a method of making DNA microarrays that employ sets of oligonucleotides having medium to long sequences (≦30 or 40 bases).

### SUMMARY OF THE INVENTION

The present invention in one aspect relates to an array for biological assays. The array comprises a solid substrate having a plurality of biosites. Each biosite having at least one set of pre-synthesized oligonucleotides of a length of about 40 bases or longer, attached to the substrate by predominantly non-covalent means. The oligonucleotides exhibits no predetermined spatial orientation upon said substrate, and are cross-linked to each other to form a network, wherein when under hybridization conditions oligonucleotide strands are free-floating and have three-dimensional freedom of movement in space.

The present invention relates also to a method for producing a microarray using chemically synthesized oligonucleotides instead of enzymatically amplified DNA. The method comprises, in part, providing a substrate and a plurality of chemically synthesized, single-stranded oligonucleotides. The each oligonucleotide strand has a length of at least about 40 bases or longer. The oligonucelotides are cross-linking to each other to form a networked complex. Networked oligonucleotides cross-link with each other, preferably, by covalent means, which in one embodiment is accomplished by UV irradiation. The complex of oligoneucleotides is affixed to a substrate by predominantly non-covalent means (e.g., electrostatic, hydrogen-bonding, hydrophobic, van der waal force, etc.). The oligonucleotide exhibits no predetermined orientation upon the substrate. It is believed that some strands of oligonucleotides, having a medium to long length, serve to anchor the complex to the substrate surface, while other strands of oligonucleotides of varying lengths serve to promote unbiased hybridization. The latter type of oligonucleotide possesses increased degrees of freedom of movement in a three-dimensional configuration under wet conditions, which more closely approximates the natural spatial orientation of genetic molecules in-vivo, for easier hybridization with target sequences. Moreover, in furtherance of unbiased or uninhibited hybridization, the method preferably uses oligonucleotides that are not chemically modified.

### BRIEF DESCIPTION OF FIGURES

FIG. 1 is a schematic representation of a typical PCR-product sequence that is bound to a substrate and undergoing hybridization with a target sample nucleic sequence.

FIG. 2 is a schematic representation of a typical short-length oligonucleotide (≦˜25-mer) with a target sample nucleic sequence floating in solution above it.

FIG. 3 is a schematic representation of the present invention according to an embodiment, wherein a layer of medium to long oligonucleotides (˜50-mer to 90mer to 180-mer) is bound to a substrate by non-covalent means, to this layer of oligonucleotides a network of other, non-modified, pre-synthesized oligonucleotides are attached in a relatively free-floating fashion to permit hybridization.

FIG. 4 shows a comparison of sensitivity and dynamic range between arrays made using PCR-product, 75-mer, size-purified 75-mer, 60-mer, size-purified 60-mer, and 30-mer sequences.

FIG. 5 is an illustration of four plots comparing the evaluation of hybridization quality and dynamic range.

FIG. 6 shows in comparison six hybridization images of array platforms for PCR-product, 75-mer, purified 75-mer (P), 60-mer, purified 60-mer (P), and 30-mer sequences, under optimized hybridization conditions.

FIG. 7 is a graph that shows signal to noise ratio of the results obtained in FIG. 7.

FIG. 8 is a graph that shows an evaluation of hybridization quality.

FIG. 9 is a chart that shows a comparison of various gene expression platforms.

FIG. 10A shows the effect of hybridization stringency on the intensity of hybridization signal of median PCR, 75-mer, purified 75-mer (P), 60-mer, purified 60-mer (P), and 30-mer.

FIG. 10B shows the quality of hybridization signal of median PCR, 75-mer, purified 75-mer (P), 60-mer, purified 60-mer (P), and 30-mer.

FIG. 11 shows a comparison of average distribution of hybridization signal in Cyanine 5 using PCR product, 75-mer, 60-mer, and 30-mer oligonucleotides.

### DETAILED DESCRIPTION OF THE INVENTION

### Definitions

Unless defined otherwise, all technical and scientific terms used herein have the meaning commonly understood by a person skilled in the art to which this invention relates. As used herein, the following terms have the meanings ascribed to them unless specified otherwise.

The term “array” or “microarray” or “DNA array” or “nucleic acid array” or “biochip” as used herein means a plurality of probe elements, each probe element comprising a defined amount of one or more nucleic acid or polypeptide molecules or elements, immobilized (including non-covalent associations, as described herein) to a solid surface or substrate.

The term “biosite” as used herein means a discrete area, spot or site on the active surface of an array, or base material, comprising at least one kind of predominantly non-covalent immobilized probe.

“Complementary” nucleic acid sequences are nucleotides on opposite strands that would normally base pair with each other.

A “target nucleic sequence” can be a chromosome or any portion thereof, or can be a recombinant nucleic acid molecule, such as a plasmid, oligonucleotide, or other nucleic acid sequence or fragment, and may be naturally occurring or synthetic. The target length is not critical provided that the target is sufficiently long to complement the probe, as described herein. When the target is DNA, it is understood that the DNA is provided for use in the method in a partially denatured or single stranded form, capable of hybridizing to a single-stranded oligonucleotide probe.

The term “solid substrate” or “substrate surface” as used in this application is a solid or “semi-solid” material, which can form a solid support for the array device of the invention. The substrate surface can be selected from a variety of materials including, for example, polyvinyl, polystyrene, polypropylene, polyester, other plastics, glass, ceramics, SiO₂, silanes, hydrogels, gold or platinum, and the like. The substrate can be in the form of a planar solid support, or an article in the form of porous or nonporous substrates, three-dimensional surfaces, beads or planar surfaces.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target sequence, and to a lesser extent to, or nor at all to, other sequences. A “stringent hybridization” and “stringent hybridization wash conditions” in the context of nucleic acid hybridization are sequence dependent, and are different under different environmental parameters.

### Description

Two currently available methods for detecting genetic polymorphisms and other types of nucleic assays use oligonucleotides and PCR-products. The present invention proposes to combine the best attributes of oligonucleotides and PCR-products in a design and method for making a DNA array. Namely, the present invention combines the freedom and specificity of oligonucleotides with the stability and good hybridzation stringency of PCR-products. One can significantly reduce the complexities of cDNA based arrays if chemically synthesized oligonucleotides of medium to long length are used as the probe material on DNA arrays, instead of an enzymatically produced large DNA fragment. Through proper definition of the parameters used in selecting oligonucleotides and the DNA sequence database, especially for gene sequences of medium to long lengths, one can also expect that an oligonucleotide based DNA array platform will deliver better performance and specificity than a cDNA based array.

The present invention provides methods for the manufacture of array devices and methods for their use in the detection and/or isolation of nucleic acids. The devices and methods according to the invention employ a predominantly non-covalent means of immobilizing nucleic acid or oligonucleotide probes. An increase in the size or length of an oligonucleotide will be useful for DNA hybridization assays when printed on solid or semi-solid, substrate surfaces. PCR typically produces DNA molecules greater than 200 base pairs, and it was thought that a DNA molecule smaller than this would not perform well in certain DNA hybridization assays. It is demonstrated, herein, that pre-synthesized, non-modified, medium to long length oligonucleotides are useful for gene expression analysis. Unlike PCR product, oligonucleotide DNA is single stranded when printed to a substrate surface. Single stranded array probes should hybridize more effectively to the solution target because there is no internal competition for hybridization.

As the length of an oligonucleotide increases, more regions on the probe oligonucleotide become available for hybridization. Theoretically, the longer the oligonucleotide is, the better target nucleic sequences will hybridize to it. Thus, an oligonucelotide with more hybridization binding sites is preferred over a shorter one. Unfortunately thus far, due to limitations of current technology, chemical synthesis of oligonucleotides that have a length greater than about 30-45-60 bases has been rather complex. And, preparation of oligonucleotides that have a length greater than about 80 bases is at the present rather difficult and inefficient, with a theoretical yield of full-length oligonucleotides at 20% or less. To overcome the synthesis limitations for extra-long oligonucleotides, to address steric concerns, and to improve the practicality of long oligonucleotides, the present invention has been developed.

An oligonucleotide of short length will attach to an amine-coated slide surface, but how available the oligonucleotide sequence is for hybridization is another issue. The present invention provides a means of making a DNA array that employs a predominately non-covalent attachment mechanism without sacrificing probe stability or stringency. This feature imparts a much easier way of manufacturing oligonucleotide DNA arrays with stable, non-covalent anchorings. Oligonucleotides can be and are randomly anchored to a substrate surface, thus no bias exists between 5′ or 3′ ends when hybridizing to a target, which can also avoid steric effects at the surface. See for comparison, a paper by T. R. Hughes et al., entitled “Expression Profiling Using Microarrays Fabricated by an Ink-jet Oligonucleotide Synthesizer,” in NATURE BIOTECHNOLOGY, Vol. 19, wpp. 342-347, April 2001, the contents of which are incorporated by reference herein. Moreover, by means of selecting an optimal oligonucleotide length (e.g., ˜65, ˜70, ˜75, or ˜85 bases) a balance between probe retention and hybridization may be struck.

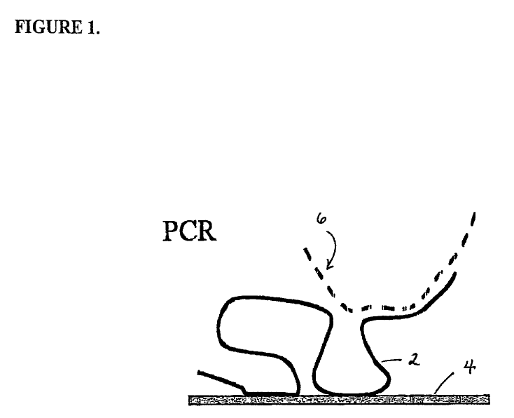

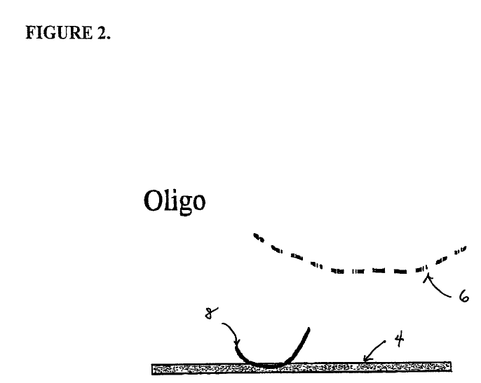

FIG. 1 shows a schematic representation of a typical PCR product (cDNA) 2 bound to a substrate surface 4 and undergoing hybridization with a target nucleic sequence 6. The length of PCR products contributes to their relative stability of attachment, while providing good hybridization sites for the target. A long strand of PCR-product has multiple regions available for hybridization and regions that anchor itself to the substrate. In comparison, FIG. 2 shows a short length oligonucleotide structure 8, of the kind predominately used in current microarray designs, adsorbed to a substrate 4, attempting to hybridize with a target sequence 6. Short length oligonucleotides cannot bind as well as longer oligonucleotide sequences with long target sequences. The inherent short length of this kind of oligonucleotide limits its hybridization effectiveness. As one can see from FIG. 2, short oligonucleotides are not able to bind to gama-amino-propyl-silane (GAPS) coated substrates as efficiently as PCR-product sequences and still have enough sequence available for hybridization. The oligonucleotide strand is limit as to good potential hybridization sites. In contrast, the present invention has a greater specificity and stringency of hybridization, since a long oligonucleotide overcomes this problem and functions in a manner similar to PCR product.

The invention comprises a “spaghetti-like” system or network of medium to long-length oligonucleotide strands. In particular, the network is formed from single stranded, pre-synthesized oligonucleotides of at least 40 bases in length. Preferably, the oligonucleotide strands each are at least 50 bases long. More preferably, the oligonucleotides have a length from about 65 to about 85 bases, but the strands can be as long as about a 125-mer, 150-mer, 180-mer or even 200-mer. An upper limit of length is only limited by available current technology in producing high quality long oligonucleotides. The oligonucleotides are preferably unmodified at either their 3′ or 5′ end. A fairly long length of oligonucleotide is exposed as potential hybridizing sites, while the entire oligonucleic structure or network is adhered securely, under common hybridization conditions, to the substrate surface by predominately non-covalent means.

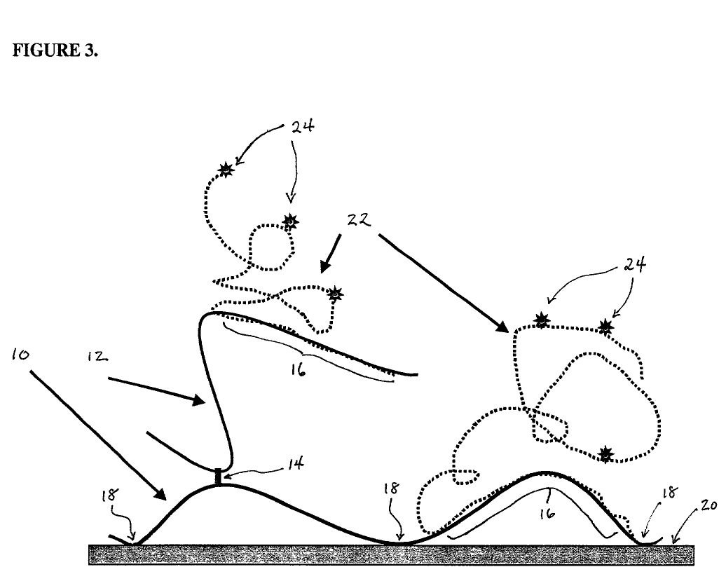

FIG. 3 is a schematic representation that depicts a section of an oligonucleotide network, according to an embodiment of the present invention, under hydrated or wet conditions. To describe and explain FIG. 3 more easily, the oligonucleotides are distinguished from each other using the terms “first oligonucleotide” or “anchoring oligonucleotide” and “second oligonucleotide.” It is believed that the following explanation reflects a theoretical understanding of oligonucleotide behavior under hybridization or other experimental conditions. This is, however, in no way intended to limit or define the actual behavior of the oligonucleotide strands relative to a substrate.

In FIG. 3, a medium to long-length oligonucleotide 10 is attached to a substrate surface by non-covalent means (e.g., electrostatic, hydrogen-bonding, hydrophobic, van der waal force, etc.) to serve as an anchor. For purpose of the present invention, the longer the strand, the stronger the oligonucleotide will bind to a selected substrate surface, hence greater stability. To this first or anchoring oligonucleotide 10, a second, non-modified oligonucleotide 12 is attached or cross-linked at an inter-oligonucleotide connection 14. Under wet conditions, the second oligonucleotide exhibits a relatively free-floating spatial orientation that is conducive to hybridization in solution. While not shown, it may be possible to attach to the second oligonucleotide a third and/or a fourth oligonucleotide strand, which is also free-floating and three-dimensionally-oriented in space under hybridization conditions. Relatively long lengths of oligonucleotide sequence 16 are exposed as potential hybridizing sites, while the entire oligonucleic structure or network is adhered securely, under common hybridization conditions, to the substrate surface by predominately non-covalent means. In other words, within the networked system, selective sections of oligonucleotides function as potential hybridization sites 16, and other sections function as anchors 18 to the substrate surface 20. Preferably, while performing hybridization assays with the present invention, each target nucleic sequence 22 is labeled with more than one dye or fluorophore label moiety 24.

In contrast to some other techniques that require fragmented, short target sequences for steric reasons, a microarray according to an embodiment of the present invention can make use of full length, multi-labeled targets, which increases the hybridization signal performance with respect to background fluorescence. The capability of increasing the number of dye markers incorporated per strand of target molecule at each binding site augments signal for each hybridized, individual, oligonucleotide strand, and the overall signal emitted from each biosite on the array. In other words, the greater the number of dye or fluorophore moieties per strand of nucleic sequence, the better the signal will likely be for improved detection and analysis. This phenomenon shows not only a better performance in respect to background fluorescence, but also a better compatibility in hybridization between a probe and target. The use of medium to long length oligonucleotides (≧60-70 bases) promotes the compatibility of probes to hybridize with targets. Oligonucleotides of medium to long lengths make it possible to use higher stringency in hybridization, which disrupts or reduces the formation of secondary structures in labeled target sequences, thereby enhancing affinity and hybridization between an oligonucleotide strand and labeled target. Further, the non-covalent attachment—as contrasted with current covalent attachment schemes—enables, the oligonucleotides to be randomly attached, without predetermined spatial orientation, to the substrate surface. This phenomenon, as mentioned before, reduces or eliminates bias between 5′ or 3′ ends when hybridizing to a target, thus avoiding steric effects at the surface.

When fabricating the network, various oligonucleotide strands are cross-linked to each other. The inter-oligonucleotide connections are either by covalent or non-covalent means, but preferably covalent. According to one method of cross-linking, the oligonucleotide strands are irradiated with ultraviolet radiation of at least about 10 m-Joules. The level of UV exposure can range from about 20 m-Joules or 30 to about 600 m-Joules. Preferably, the level of energy employed is from about 130 m-Joules to about 200 m-Joules. Covalent attachment between oligonucleotides and the substrate is limited at lower energy levels, below about 300 m-Joules, which permits in-situ oligonucleotide network formation on the surface of a substrate. (Contrary to common belief, ultraviolet cross-linking of oligonucleotides does not necessarily create covalent attachments to the underlying substrate. UV-cross-linked arrays of size-purified 75-mer oligonucleotides can be stripped nearly completely from a GAPS coated slide, when treated under conditions of 100 mM NaOH, 0.1% SDS at 85-90° C. for about 10 minutes, then rinsed with dH₂O and dried.) In another approach, bio-functional polymers, such as poly(styrene-alt-maleic anhydride)(SMA), poly(methyl-vinyl etheralt-maleic anhydride) (PVMA), poly-acrylic acid, poly-aspartic acid, polyglutamic acid, etc., are employed to cross-link the oligonucleotide strands.

The present invention affords researchers the freedom to use oligonucleotides of any length that would enable the oligonucleotide to be associated securely to a substrate and still have a relatively long sequence for hybridizing with a target nucleic sequence. Preferably, the length is greater than about 50 bases; ranging from about 65 to about 90 bases. Of course, other oligonucleotide sequences of about 95 to about 10 bases or greater in length also may be used. The oligonucleotide strands may have the same or different nucleic sequences, and/or the same length or different lengths in various combinations, depending on the desired application or use. Neither the particular oligonucleotide length nor specific nucleic sequence is a limiting factor of the invention. For instance, an anchoring oligonucleotide, like the one labeled 10 in FIG. 3, which binds non-covalently with a substrate 20, may have a nonsensical sequence that does not encode for any real gene. The real and desired sequence for hybridization would be located in a second and/or third oligonucleotide, rather than in the anchor oligonucleotide. In such a situation, the anchoring oligonucleotide would not complicate hybridization of target nucleic sequences, and can decrease the background noise level.

Labeled target nucleic sequences 22 having relatively long, complementary segments, will successfully hybridize with sections of the network of free-floating, three-dimensionally oriented oligonucleotide probes, without the need for fragmentation, as would be required in short oligonucleotide array platforms. Hence, the present invention would permit researchers to study long gene sequences that usually can not readily be expressed by existent oligonucleotide techniques. Preferably, while performing hybridization assays with the present invention, each target nucleic sequence is labeled with more than one dye label moiety. To reiterate, in contrast to some other techniques that require fragmented, short target sequences for steric reasons, a microarray according to an embodiment of the present invention can make use of full length, labeled targets, which increases the hybridization signal performance with respect to background fluorescence. With a longer probe, longer target sequences having an increase signal per labeled target can be used. Increasing the number of dye markers incorporated per strand of target molecule at each binding site augments signal for each hybridized, individual, oligonucleotide strand, and the overall signal emitted from each biosite on the array.

This phenomenon shows not only improved performance with respect to background fluorescence, but also better compatibility between probe and target in hybridization. When medium to long-length oligonucleotides are used, longer gene sequences will more likely hybridize completely with target sequences. A medium to long length oligonucleotide strand is more easily able to expose binding sites in the target, hence eliminating the problems that short oligonucleotides have with secondary structures in the target strands. The binding stringency and secondary structure stringency are matched. Oligonucleotide strands of increased lengths make it possible to use higher stringency in hybridization, which disrupts or reduces the formation of secondary structures in labeled target sequences, thereby enhancing affinity and hybridization between an oligonucleotide strand and labeled target.

Another virtue of the present invention relates to the ease of manufacture and design of microarrays. Chemically, synthesized sequences are liberated from dependence on cDNA templates. If one were to use chemically synthesized oligonucleotides, rather than PCR products, every conceivable DNA sequence becomes available, and with greater specificity. Subject only to the sophistication of oligonucleotide-selection computer software, one can design virtually any gene sequence or combination of specific sites on an oligonucleotide strand, and avoid repetitive homologues which may lead to problems of cross hybridization and high background signal.

Methods for manufacturing an array of networked oligonucleotides are varied. The oligonucleotide networks can be formed either in-situ on a substrate or in solution, with subsequent deposition on the substrate. Generally, a method comprises: providing a plurality of single-stranded oligonucleotides, each having a length of 40 bases or greater; cross-linking the oligonucleotides to form a network; providing a substrate; depositing the network onto the substrate, wherein the network is attached to the substrate by a predominantly non-covalent means. For in-situ network formation from pre-synthesized oligonucleotides, a first or anchoring oligonucleotide is layed-down and attached non-covalently to the substrate. A second- or third and/or fourth oligonucleotide strand with the same or different length and/or with the same or different sequence as the first oligonucleotide is cross-linked covalently to the first oligonucleotide, as described and illustrated above. Alternatively, another method comprises: providing a substrate; providing a solution of single-stranded oligonucleotides of varying lengths; cross-linking the oligonucleotide strands to each other to forming a network; and depositing the solution to the substrate. The oligonucleotides are cross-linked in solution before deposition or printing onto a prepared substrate.

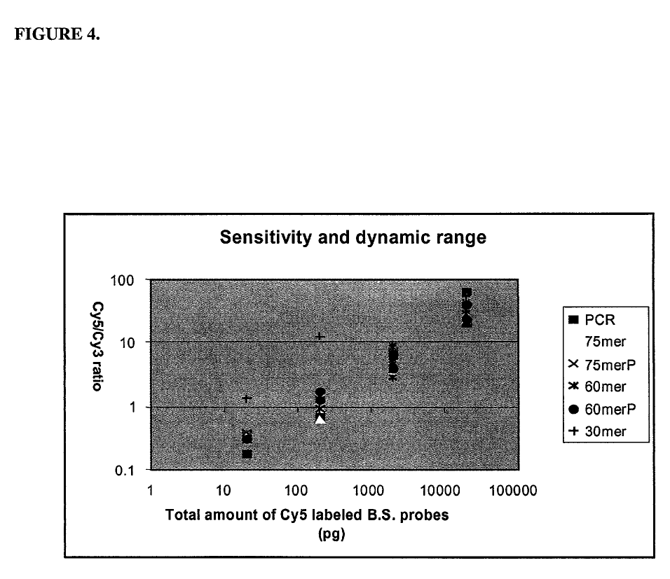

Furthermore, the invention demonstrates that oligonucleotides that are at least about 40 bases long or longer (about 50-60-75-80-100-150-180-220 bases, etc.) can perform as well as PCR products in detecting low level gene expression at a level of 10 pg of labeled cDNA. As can be seen from accompanying FIGS. 4, 6, and 8, pre-synthesized 30-mers, 60-mers, and 75-mers printed on a solid substrate hybridize with complex cDNA yeast samples just as well as PCR products do. Each oligomer sample produces an expression profile to similar that attained from PCR products. A longer oligomer, it appears functions better. Thus, single stranded or networked-complexes of medium or long-length oligonucleotides or oligonucleotide dendrimers could replace PCR products as the biological material of choice for DNA microarray preparation.

In a series of experiments, the details of which are given below, the feasibility of a non-modified oligonucleotide based DNA arrays were explored on gamma-aminopropyl-silane (GAPS) coated glass slides, according to one embodiment. Results indicate that oligonucleotide networks can act in a manner similar to the PCR product, with the same or better levels of performance as present PCR-product-based DNA arrays. A comparison of networks containing oligonucleotides with a length from about 45 to about 85 nucleotides, after UV cross-linking, to PCR product with a 500 bp length showed no significant difference in the sensitivity, dynamic range, linear range of a two-color ratio measurement, distribution of hybridization signal, and signal-to-noise level.

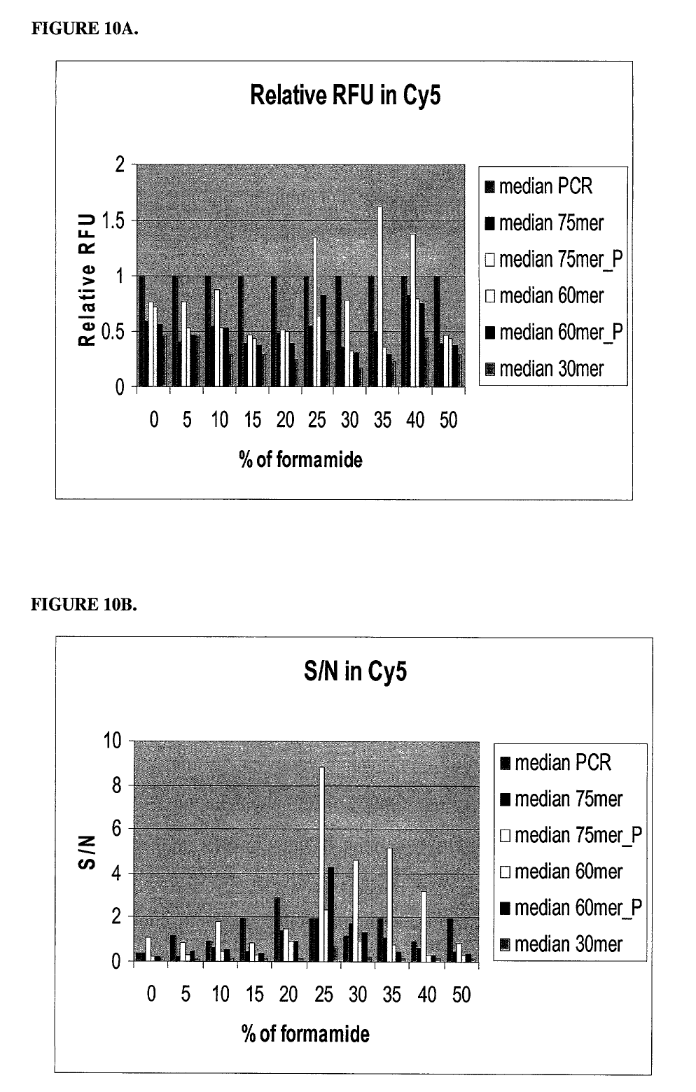

The effects of hybridization stringency on the performance of oligonucleotide and PCR platforms were also investigated. Ten 100 gene array slides were prepared to test 10 different hybridization buffers. FIGS. 10A and 10B summarize the results of the tests. FIGS. 10A and 10B, respectively, illustrate the effect of hybridization stringency on the intensity of hybridization signal and the quality of the signal. The concentration of formamide employed was varied (0%, 5%, 10%, 15%, 20%, 25%, 30%, 35%, 40% and 50%) to adjust the hybridization stringency. All other components of the hybridization buffer remained constant: 5× SSC, 0.1% SDS and 10 μg/ml herring sperm DNA. The slides were probed with lung total RNA in Cyanine 3 (Cy3) and testis total RNA in Cyanine 5 (Cy5).

Of all the platforms tested, size-purified 75-mers seem to give a much better signal and signal to background noise (S/N) ratio than any of the other samples at an optimized hybridization stringency of about 24% to about 40% formamide. For a 60-mer the optimal stringency, according to the same test, was from about 15% to about 30% formamide. The experimental results appear to be even better than the values obtained for median PCR samples, as can been seen in FIGS. 10A and 10B using Cy5.

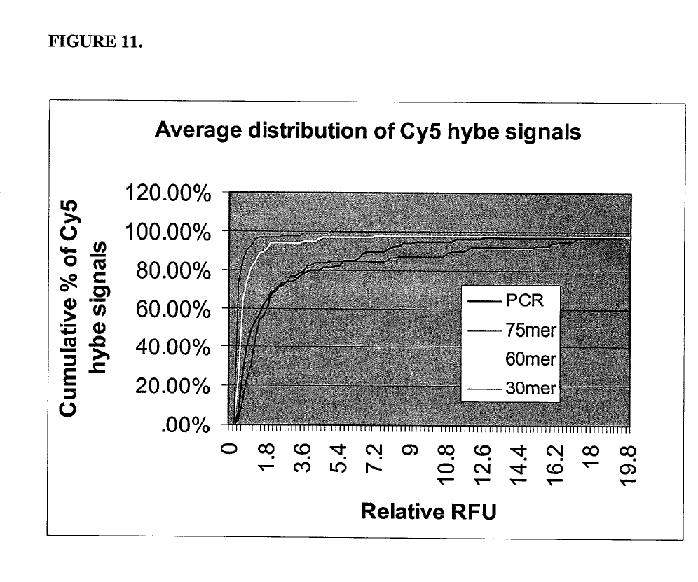

The relative performance of size-purified 75-mer oligonucleotides is comparable to PCR product on gamma-amino-propyl-silane (GAPS) coated slides after UV-cross linking. Both sensitivity and dynamic range on oligonucleotide arrays with oligonucleotide lengths greater than about 60-mer are comparable to cDNA probes. The hybridization signal intensity and their distribution on purified 75-mer oligonucleotide array is nearly indistinguishable to a cDNA array, as seen in FIG. 11. Differential gene expression was observed for all oligonucleotides samples, even as short as 30-mers. With real-time PCR method, one is able to confirm the differentially expressed genes with a greater than 2 to 3 fold of change in the gene expression level. Thus, oligonucleotides that have a length above a minimal length of about 40-45 bases can offer another viable option for fabricating DNA microarrays without compromising the array performance. Preferably the oligonucleotides have about 50-mer to about 60-mer. More preferably the oligonucleotides have a length from about 70-mer to about 85-mer, or from about 80-mer to 100-mer to 150-mer.

Combinations of medium and long oligonucleotides of various lengths and/or sequences in a network could be applied together. An economic use of medium length and very long oligonucleotide sequences permits the invention to overcome the problems associated with mass syntheses of large oligonucleotides. One can avoid the associated chemical and steric problems, as well as the time and expense related to production that others have encountered. Furthermore, the invention permits a more naturalistic binding of medium length oligonucleotides to a long oligonucleotide that serves to anchor by non-covalent means the networked mass of oligonucleotides to a substrate. The mass of cross-linked oligonucleotides forms a multi-layer on the surface of the substrate.

### Experiments

According to the present invention, one fabricated arrays using oligonucleotides of varying sizes and purity on a gamma-amino-propyl-silane (GAPS) coated slide. Medium-length oligonucleotides, such as those referred to in FIG. 1, in the range of about 50 to about 85 bases, were purified or enriched and used to construct an interconnected network of oligonucleotides by means of UV cross-linking the individual oligonucleotides to each other. The performance of oligonucleotides with a length great than 40-mer was found to be comparable to PCR product on GAPS slides after UV cross-linking. Both sensitivity and dynamic range on oligonucleotide arrays with oligonucleotide-lengths greater than 40-mer are comparable to cDNA targets, although differential gene expression was observed for all oligonucleotides, including a 30-mer. One was able to confirm the expression level of differentially expressed gene with a real-time PCR method. Oligonucleotides from about 30 to about 85 nucleotide length can offer another option for fabricating DNA microarray on Corning GAPS slide without compromising the array performance.

To test and benchmark the utility of non-modified median size oligonucleotides as probes on DNA array fabricated on GAPS coated slide surface, oligonucleotides were printed at a concentration of 0.5 μg/μL in 3× SSC and PCR products were printed in ink containing 50% DMSO at the concentration of 0.25 μg/μL. A 3×2 grid pattern was used to print the slide on a Cartessian Technologies robot using Telechems “Stealth” quill pins and Corning™ GAPS coated slides. The printed slides were dried and stored in a desiccator. Oligonucleotides on the slides were cross-linked with 300 mJ in a Stratagene UV Stratalinker 2400 before use.

A 100-gene array that composed of 85 well-known human genes with diverse functions, 7 housekeeping genes, and 8 Bacilus subtilis control genes was designed. Three sets of 100 oligonucleotides of different lengths were chemically synthesized and desalted. Aliquots of the 75-mers and 60-mers were subjected to PAGE purification to enrich the full-length oligonucleotides. More than 90% of the oligonucleotide in the size-purified samples were full-length oligonucleotides. For the same 100 genes, a set of DNA probes with lengths of 500 base pairs was generated by PCR amplification method. On this second array, one spotted 6 different types of DNA for each gene.

The experiment employed different lengths of oligonucleotides at sizes of about 30-mer, 60-mer, and 75-mer. Size-purified 60-mer and 75-mer sequences were spot-deposited on the slide along with the unpurified counterparts. Besides these five types of oligonucleotides, the PCR products of the 3′ UTR of the “probe” genes were also spotted on the same slide as the control for the benchmarking experiments. The experiment evaluated six key attributes of these expression arrays which included: sensitivity, dynamic range of the hybridization signals, linear range of the two-color ratio, the distribution of the hybridization signals, the quality of the hybridization signals and the ability to detect the differential gene expression (DGE) patterns.

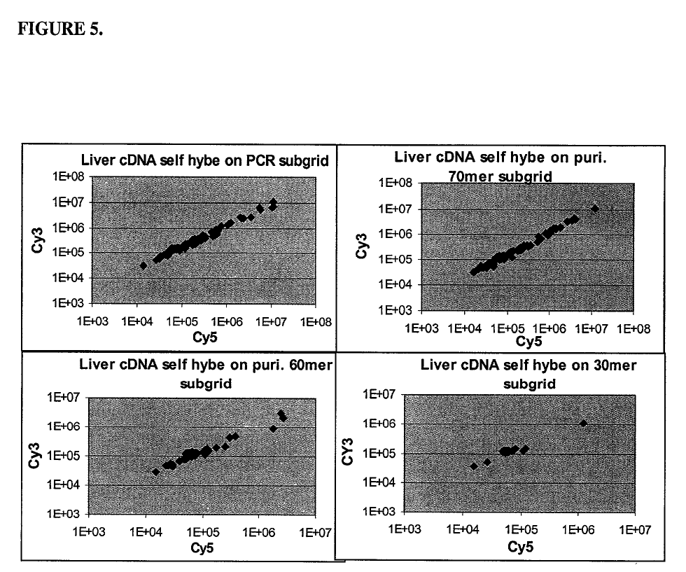

To examine the sensitivity and the linear range of two-color ratio measurements, we labeled each of 8 bacteria B. subtilis control genes with both Cyanine 3 (Cy3; green) and Cyanine 5 (Cy5; red) dyes, and hybridized them to the DNA arrays. In this hybridization, equal amount (5.5 ng) of cRNA of the eight control genes were mixed and used in the labeling reaction to generate Cy3 dye labeled probes. For Cy5 labeled probes, a cRNA mixture was made with 20 ng of two controls 1 and 2; 2 ng of another two controls 3 and 4; 0.2 ng of two controls 5 and 6; 0.02 ng of two other controls 7 and 8. The Cy3 and Cy5 labeled probes were mixed and hybridized to the array. FIG. 5 shows results for control genes. The Y-axis is the Cy5/Cy3 ratio of hybrid signals and the X-axis is the ratio of the Cy5 and Cy3 labeled probe concentration. As mentioned before, the amount for Cy3 probes for 8 genes is fixed, however, the amounts of Cy5 probes were at 10-fold serial dilution (20 ng: 2 ng: 0.2 ng: 0.02 ng). The result indicates that there is good linear relationship between the ratio of the Cy3 and Cy5 probe concentration and the measured Cy3/Cy5 hybridization signal ratios. The linear range is over 3 logs of probe concentration difference on both oligonucleotide and PCR product arrays. Since the lowest concentration of the Cy5 labeled probes are at 20 pg of cRNA/slide, we estimated that the detection limits for both PCR and oligonucleotide arrays were at 10 copy of mRNA per cell level.

One used the Cy3 and Cy5 labeled total RNA from human liver tissue for the self-self hybridization to evaluate the dynamic range and the distribution of the Cy3 and Cy5 ratios. FIG. 5 shows four scatter-plots of the fluorescent signal from each biosite spot after quantification. Cy3- and Cy5-labeled probes were generated from equal amounts of human liver total RNA. The equal distant distribution of the points between the X and Y axes shows that there is no significant variation in the hybridization signals between Cy3- and Cy5 -labeled probes prepared from the same RNA source on both oligonucleotide and PCR arrays. One observed a very tight distribution of the two-color ratios within the slide (CV<10%). One estimated that the dynamic range of the hybrid signals to be at 2.5 to 3 logs. This does not limit the useful range for determination of ratios in transcript abundance using a two-color (i.e., red/green) system, because identical transcripts will compete even at saturating concentrations, preserving transcript ratios.

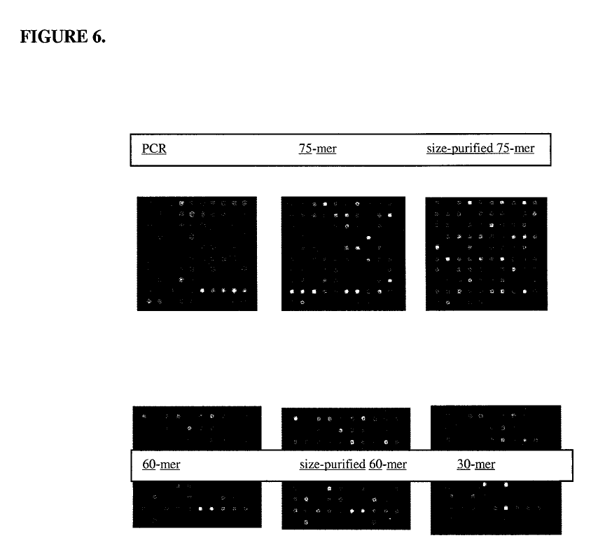

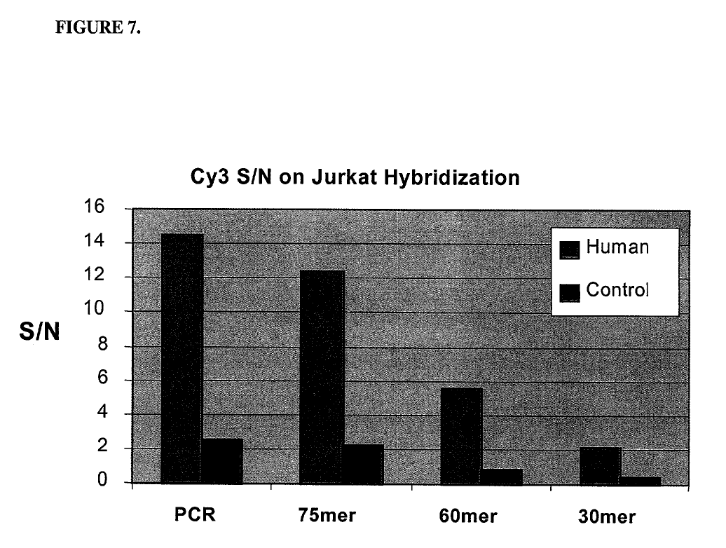

In order to compare the hybridization signal intensities, specificity and the quality of the hybridization signals on different array platforms as well as the ability to detect differential gene expression pattern (DGE), four sets of DGE experiments were carried out. FIG. 6 illustrates the hybridization images of the DGE experiment under optimized hybridization conditions for each array platforms. FIG. 7 shows a graph of the plot for the average signal-to-noise of the extracted hybridization signal. The lighter colored column (left) is S/N of human genes and the darker colored (right) column represents the S/N of control genes. The data in FIG. 7 suggests that the hybridization was specific: significant higher signal was observed on human targets than on control genes. Also, at the optimized hybridization conditions, all oligonucleotide array platforms yielded reasonable hybridization signals, and the intensity of the hybridization signal was DNA size dependent. That is, higher hybridization signals were observed on PCR and 75-mer than on 60-mer and 30-mer arrays.

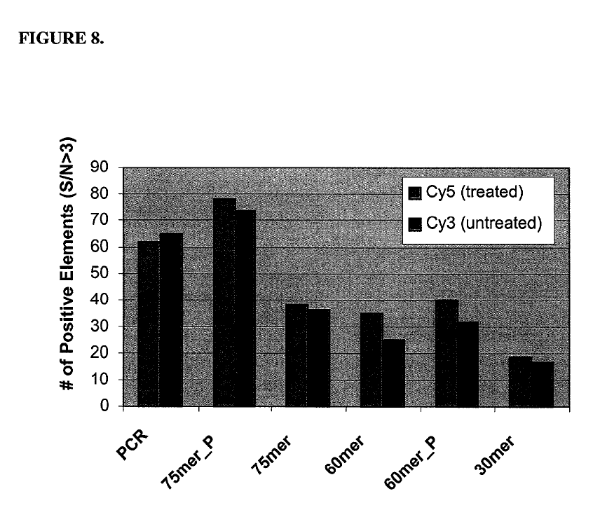

The experiment also examined the quality of the hybridization signals. FIG. 8 present the number of positive elements or biosites with signal-to-noise ratio of greater than three (3). Consistently, a size-purified 75-mer oligonucleotide array exhibited a better quality of signal to noise ratio, of about 20% more of the array elements, than even a PCR product array.

One of the main concerns regarding oligonucleotide platforms is the ability of the oligonucleotides to detect the difference of mRNA level in a DGE experiment and the consistency between an oligonucleotide platform and a PCR platform. According to the findings, similar expression profiles from both PCR and oligonucleotide platforms were observed. A 75-mer oligonucleotide array and a PCR array were able to generate an overlap of about five (5) genes that were scored as differentially expressed genes in human lung and testis based on the 2-fold +/−1 SD cutoff criteria.

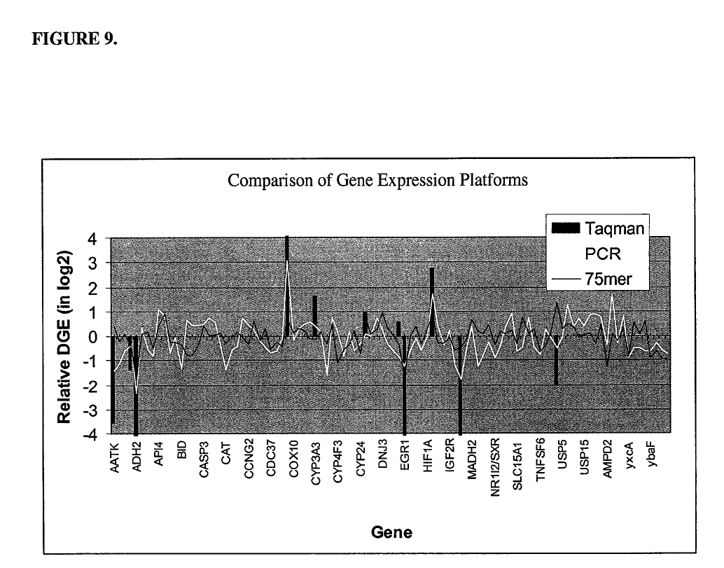

From the 100-gene test array, 11 genes were selected, which included all five (5) genes that were scored as differentially expressed in human lung and testis for confirmation by the real-time PCR method (Taqman-PE Biosystem ABI Prism 7700 Sequence Detection System). The progress of the real-time PCR reaction was monitored by the binding of the SybrGreen dye to the resulting dsDNA product. The relative abundance of the mRNA was then estimated based on the difference of the Ct values and efficiency of the PCR amplification. As shown in FIG. 9, all the genes scored as DGE genes by both oligonucleotide and PCR array platforms shown similar trends of the differential gene expression and those trends were confirmed by the real-time PCR reactions.

Although the present invention has been described by way of examples, it will be understood by those skilled in the art that the invention is not limited to the embodiments specifically disclosed, and that various modifications and variations can be made without departing from the spirit and scope of the invention. Therefore, unless changes otherwise depart from the scope of the invention as defined by the following claims, they should be construed as included herein.

We claim:

1. An array for biological assays comprising:
a solid substrate having a plurality of biosites;
each biosite having at least one set of pre-synthesized oligonucleotides, each having a length of about 40 bases or longer, attached to said substrate by predominantly non-covalent means;
said oligonucleotides are cross-linked to each other to form a network.

2. The array according to claim 1, wherein said oligonucleotides exhibits no predetermined spatial orientation upon said substrate

3. The array according to claim 1, wherein said oligonucleotides are unmodified.

4. The array according to claim 1, wherein said oligonucleotides have a length in a range from about a 40-mer to about a 85-mer

5. The array according to claim 3, wherein said oligonucleotides have a length in a range from about a 50-mer to about a 80-mer.

6. The array according to claim 1, wherein said oligonucleotides have a length in a range from about a 80-mer to about a 150-mer.

7. The array according to claim 1, wherein said substrate has a surface made from a material selected from the following: plastic, glass, metal, or composite materials.

8. The array according to claim 1, wherein said substrate has a surface made from a material selected from the following: polyvinyl, polystyrene, polypropylene, polyester plastics.

9. The array according to claim 1, wherein said substrate has a surface made from glass.

10. The array according to claim 1, wherein said substrate has a surface constituent selected from SiO₂, silanes, hydrogels, gold, or platinum coatings.

11. The array according to claim 1, wherein said oligonucleotide strands are free-floating and have three-dimensional freedom of movement in space when under hybridization conditions

12. An array comprising:
a) a multi-layered network of cross-linked oligonucleotide strands,
b) each of said strands having a length of about 50 bases or greater,
c) said oligonucleotide strands are associated to a substrate by non-covalent means,
d) such that said oligonucleotide strands exhibits no predetermined orientation upon said substrate.

13. The array according to claim 12, wherein said oligonucleotide strands have a length of in a range from about 65 bases to about 85 bases.

14. The array according to claim 12, wherein said oligonucleotides have a length in a range from about 85 bases to about 95 bases.

15. The array according to claim 12, wherein said oligonucleotides have a length of about 100 bases to about 180 bases.

16. The array according to claim 12, wherein said substrate does not possess an electrostatic charge.

17. The array according to claim 12, wherein said oligonucleotide strands are chemically unmodified.

18. The array according to claim 12, wherein said oligonucleotide strands exhibit relatively natural conformations as observed in-vivo under fluidic hybridization conditions.

19. A method for fabricating microarrays, the method comprising:
a) providing a plurality of pre-synthesized oligonucleotide strands, each having a length of about 40 bases or greater;
b) cross-linking said oligonucleotide strands to form a network;
c) providing a substrate;
d) affixing said network to a substrate by a non-covalent means.

20. The method according to claim 19, wherein said oligonucleotide strands are affixed in a manner that exhibits no predetermined orientation upon said substrate.

21. The method according to claim 19, wherein said oligonucleotide strands are cross-linked covalently with each other.

22. The method according to claim 19, wherein said at least one oligonucleotide strand anchors said network to said substrate.

23. The method according to claim 19, wherein a number of said oligonucleotide strands exhibit a three-dimensional freedom of movement under wet conditions.

24. The method according to claim 19, wherein said substrate possesses an electrostatic charge.

25. The method according to claim 19, wherein said oligonucleotide strands are unmodified.

26. The array according to claim 19, wherein said oligonucleotides have a length in a range from about a 50-mer to about a 85-mer

27. The array according to claim 26, wherein said oligonucleotides have a length in a range from about a 65-mer to about a 80-mer.

28. The array according to claim 19, wherein said oligonucleotides have a length in a range from about a 80-mer to about a 150-mer.

29. The array according to claim 19, wherein said substrate has a surface made from a material selected from the following: plastic, glass, ceramic, metal, or composite materials.

30. The array according to claim 19, wherein said substrate has a surface made from a material selected from the following: polyvinyl, polystyrene, polypropylene, polyester plastics.

31. The array according to claim 19, wherein said substrate has a surface made from glass.

32. The array according to claim 19, wherein said substrate has a surface constituent selected from SiO₂, silanes, hydrogels, gold, or platinum coatings.

33. A method for fabricating microarrays, the method comprising:
a) providing a first oligonucleotide having a length of about 40 bases or greater,
b) crosslinking said first oligonucelotide with a chemically unmodified, single-stranded, second oligonucleotide to form a network,
c) affixing said network to a substrate by predominantly non-covalent means.

34. The method according to claim 33, wherein said first oligonucleotide has a length of about 50 nucleotides or greater, and is affixed directly to said substrate.

35. The method according to claim 34, wherein said first oligonucleotide has a length of in a range from about 100 nucleotides to about 150 nucleotides.

36. The method according to claim 33, wherein said second oligonucleotide has a length of about 65 nucleotides or greater.

37. The method according to claim 36, wherein said second oligonucleotide has a length in a range from about a 75-mer to about a 150-mer.

38. The method according to claim 33, wherein said second oligonucleotide is free-floating and has three-dimensional freedom of movement in space when under hybridization conditions.

39. The array according to claim 33, wherein said substrate has a surface made from a material selected from the following: plastic, glass, ceramic metal, or composite materials.

40. The array according to claim 33, wherein said substrate has a surface made from a material selected from the following: polyvinyl, polystyrene, polypropylene, polyester plastics.

41. The array according to claim 33, wherein said substrate has a surface made from glass.

42. The array according to claim 33, wherein said substrate has a surface constituent selected from SiO₂, silanes, hydrogels, gold, or platinum coatings.

43. A method for detecting genetic polymorphism, the method comprises:
a) providing an array comprising a number of biosites with a network of cross-linked oligonucleotides, in which a number of said networked oligonucleotides are non-covalently associated with a substrate;
b) hybridizing target nucleic acid sequences to said first or second set of oligonucelotides to attain a hybridization sensitivity of comparable quality as that achieved with polymerase chain reaction products.

44. The method according to claim 43, wherein said oligonucleotides each having a length of about 50 bases or longer.

45. The method according to claim 43, wherein said oligonucleotides each is chemically unmodified.
